(19) United States
(12) Patent Application Publication
(10) Pub. No.: US 2026/0366076 A1
(43) Pub. Date: Nov. 19, 2026

(54) METHODS AND SYSTEMS FOR ISOLATING NUCLEIC ACIDS

(71) Applicant: Foundation Medicine, Inc., Boston, MA (US)

(72) Inventors: Lauren Nicole Mach QUIGLEY, Malden, MA (US); Benjamin MCNALLY, Westwood, MA (US)

(73) Assignee: Foundation Medicine, Inc., Boston, MA (US)

(21) Appl. No.: 19/137,592

(22) PCT Filed: May 3, 2024
(86) PCT No.: PCT/US2024/027770

§ 371 Date: Jun. 10, 2025

Related U.S. Application Data
(60) Provisional application No. 63/464,484, filed on May 5, 2023.

(51) Int. Cl. C12Q 1/6874; C12N 15/10; C12Q 1/6806; C12Q 1/6855

(52) U.S. Cl. CPC C12Q 1/6874; C12N 15/1013; C12Q 1/6806; C12Q 1/6855

(57) ABSTRACT

Described herein is a method for isolating nucleic acid molecules. The method can comprise: removing proteins from a solution comprising nucleic acid molecules; adding a substrate and a binding reagent comprising an alcohol to the solution after removing the proteins from the solution to bind the nucleic acid molecules to the substrate; and separating the substrate bound to the nucleic acid molecules from a remainder of the solution.

### CROSS-REFERENCE TO RELATED APPLICATION

This application claims the priority benefit of U.S. Provisional Patent Application Ser. No. 63/464,484, filed May 5, 2023, the contents of which are incorporated herein by reference in their entirety.

### FIELD OF THE DISCLOSURE

The present disclosure relates generally to methods and systems for isolating nucleic acids, and more specifically, to methods and systems for isolating nucleic acids for preparing a nucleic acid sequencing library.

### BACKGROUND

Nucleic acid sequencing has increasingly become a significant tool for diagnosing, monitoring or determining the appropriate treatment for disease, such as cancer. To address this demand for higher sequencing throughput, it is increasingly important to develop automated nucleic acid extraction and/or isolation protocols and systems, while also maintaining high isolation efficiency to retain as much of the nucleic acid molecule present in a sample for downstream processing (e.g., library construction) and sequencing.

Some studies have published methods using phenol: chloroform: isoamyl alcohol (PCI) to isolate single stranded(ss) and double stranded (ds) cell free DNA from plasma. This extraction approach relies on phase separation where organic molecules are solubilized in phenol, polar molecules such as DNA are retained in the upper aqueous phase, and proteins, which have polar and non-polar residues, separate the layers, constituting the interphase layer. The DNA is then precipitated out of the aqueous solution by adding salt and alcohol.

The salt neutralizes the negative charge along the DNA backbone making DNA less soluble in water, thus allowing the DNA to be isolated by centrifugation. Hisano et al., Short single-stranded DNAs with putative non-canonical structures comprise a new class of plasma cell-free DNA, BMC Biol., vol. 19, no. 225(2021 ), used a traditional phenol: chloroform: isoamyl alcohol extraction followed by isopropanol precipitation to extract ssDNA from plasma alongside dsDNA. Cheng et al., Plasma contains ultrashort single-stranded DNA in addition to nucleosomal cell-free DNA, iScience, vol. 25, no. 104554(2022 ), used a similar approach where they used phenol: chloroform: isoamyl alcohol to isolate the aqueous phase of plasma. They then used solid-phase reversible immobilization (SPRI) beads to isolate DNA from the aqueous phase before extracting the DNA with an ethanol precipitation. These approaches, however, are not readily automatable. Phenol is a corrosive compound with a low viscosity, which contributes to it being a drip risk, when incorporated into automated systems. The inadvertent dripping of phenol can result in instrumentation damage. Further, given the low viscosity of phenol, the positioning of phase layers comprising phenol is highly inconsistent, across, and even within, samples. As a result, instructing an automated system, such as a liquid handler, to intake a specific volume is challenging and often inaccurate. Further, phenol is a volatile organic compound that has health and safety risks associated with its use. Approaches that do not use phenol would improve the overall useability.

### BRIEF SUMMARY OF THE INVENTION

Disclosed herein are methods and systems for isolating nucleic acids from a biological sample. The methods for isolating nucleic acid molecules include removing proteins from the sample prior to isolating the nucleic acids from the sample, which allows the process to be more readily automatable and avoids the use of hazardous solvents such as phenol. The early removal of proteins from the sample, relative to existing methods in the art, enables a phenol-free method of isolating nucleic acid molecules. Avoiding the use of phenols is conducive to scaling and automating nucleic acid isolation methods. The scalable method of nucleic acid isolation disclosed herein can be used for isolating cell-free single stranded DNA (ssDNA), including ultra-short ssDNA fragments, from the sample. The sample from which the nucleic acid molecules are isolated can comprise of human (or non-human) plasma samples, and the nucleic acid molecules can be analyzed for downstream diagnoses and prognoses of diseases, such as cancer. In summary, the potential applications of the methods disclosed herein, such as the automatable and scalable clinical analyses of cell-free ssDNA from plasma samples, ultimately stems from the present method's early removal of proteins from the sample.

A method of isolating nucleic acid molecules, which may be automated, can include: removing proteins from a solution comprising nucleic acid molecules; adding a substrate and a binding reagent comprising an alcohol to the solution after removing the proteins from the solution to bind the nucleic acid molecules to the substrate; and separating the substrate bound to the nucleic acid molecules from a remainder of the solution. In some embodiments, the substrate and the binding reagent can be added simultaneously to the solution. In some embodiments, the nucleic acid molecules can be derived from a liquid sample. In any of the embodiments herein, the nucleic acid molecules can comprise single-stranded DNA, double-stranded DNA, and/or RNA. In some embodiments, the RNA can be mRNA.

In any of the embodiments herein, the disclosed methods can further comprise adding a proteinase to the solution prior to removing proteins from the solution. In some embodiments, the proteinase can be proteinase K or trypsin. In any of the embodiments herein, the disclosed methods can further comprise adding a detergent to the solution prior to removing the proteins from the solution. In some embodiments, the detergent can comprise nonyl phenoxypolyethoxylethanol (NP-40), radio-immunoprecipitation assay (RIPA), sodium dodecyl sulfate (SDS), ammonium-chloride-potassium (ACK), or p-(2,4,4-trimethylpentan-3-yl)phenyl ether (Triton). In any of the embodiments herein, removing proteins from the solution can comprise precipitating the proteins. In some embodiments, precipitating the proteins can comprise adding a chaotropic agent or an anti-chaotropic agent to the solution.

In some embodiments, the chaotropic agent can be a chaotropic salt. In any of the embodiments herein, the chaotropic agent can be sodium chloride, urea, guanidine thiocyanate, lithium acetate, or sodium thiocyanate. In any of the embodiments herein, the anti-chaotropic agent can comprise ammonium sulfate or Zn²⁺ ions.

In any of the embodiments herein, precipitating the proteins can comprise centrifuging the proteins into a pellet. In some embodiments, the methods disclosed herein can further comprise separating a supernatant comprising the nucleic acid molecules from the pellet. In any of the embodiments herein, the binding reagent can comprise a chaotropic agent. In some embodiments, the chaotropic agent can comprise ammonium ions, potassium ions, sodium ions, lithium ions, magnesium ions, calcium ions, guanidium ions, fluoride ions, sulfate ions, phosphate ions, acetate ions, chloride ions, bromide ions, nitrate ions, chlorate ions, thiocyanate ions, or any combination thereof. In any of the embodiments herein, the binding reagent can comprise a polysaccharide or polyethylene glycol (PEG). In some embodiments, the binding reagent can comprise the polysaccharide, and wherein the polysaccharide is dextran.

In any of the embodiments herein, the alcohol can comprise isopropanol. In any of the embodiments herein, the alcohol can comprise ethanol. In any of the embodiments herein, the substrate can comprise beads. In some embodiments, the beads can be solid phase reversible immobilization (SPRI) beads. In any of the embodiments herein, the beads can be silica beads. In any of the embodiments herein, the beads can be magnetic beads. In some embodiments, separating the substrate bound to the nucleic acid molecules from the remainder of the solution can comprise applying a magnetic field to the magnetic beads. In any of the embodiments herein, the substrate can comprise or can be coated with silica.

In any of the embodiments herein, the methods disclosed herein can further comprise separating nucleic acid molecules from the substrate after the substrate has been separated from the remainder of the solution. In some embodiments, the nucleic acids molecules can be separated from the substrate by dissolving the nucleic acid molecules in a solvent. In some embodiments, the solvent can be water or a Tris-EDTA (TE) buffer solution. In any of the embodiments herein, the solution can comprise plasma, whole blood, buffy coat, saliva, serum, sputum, stool, or cerebrospinal fluid.

In any of the embodiments herein, the methods disclosed herein can further comprise: providing the nucleic acid molecules obtained from a sample from a subject; ligating one or more adapters onto one or more nucleic acid molecules from the nucleic acid molecules; amplifying the one or more ligated nucleic acid molecules from the nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; and receiving, at one or more processors, sequence read data for the plurality of sequence reads. In any of the embodiments herein, the one or more adapters can comprise amplification primers, flow cell adaptor sequences, substrate adapter sequences, or sample index sequences. In any of the embodiments herein, the captured nucleic acid molecules can be captured from the amplified nucleic acid molecules by hybridization to one or more bait molecules. In some embodiments, the one or more bait molecules can comprise one or more nucleic acid molecules, each comprising a region that is complementary to a region of a captured nucleic acid molecule. In any of the embodiments herein, amplifying nucleic acid molecules can comprise performing a polymerase chain reaction (PCR) amplification technique, a non-PCR amplification technique, or an isothermal amplification technique. In any of the embodiments herein, the sequencing can comprise use of a massively parallel sequencing (MPS) technique, whole genome sequencing (WGS), whole exome sequencing, targeted sequencing, direct sequencing, Nanopore sequencing technique or Sanger sequencing technique. In some embodiments, the sequencing can comprise massively parallel sequencing, and the massively parallel sequencing technique comprises next generation sequencing (NGS). In any of the embodiments herein, the sequencer can comprise a next generation sequencer.

Disclosed herein is a method of detecting a genetic variant, comprising: sequencing the isolated nucleic acid molecules according to any of the embodiments disclosed herein to obtain a plurality of sequence reads; and calling, using one or more processors, the genetic variant based on the plurality of sequence reads. Disclosed herein is a method of detecting the presence of cancer, comprising: detecting a genetic variant according to some embodiments, wherein the genetic variant is indicative of a cancer. Disclosed herein is a method for monitoring cancer progression or recurrence in a subject, the method comprising: detecting a genetic variant using first isolated nucleic acid molecules in a first sample obtained from the subject at a first time point according to some embodiments; detecting the genetic variant using second isolated nucleic acid molecules in a second sample obtained from the subject at a second time point; wherein the first time point is before or after the second time point.

Disclosed herein is a method for monitoring cancer progression or recurrence in a subject, the method comprising: detecting a genetic variant using first isolated nucleic acid molecules in a first sample obtained from the subject at a first time point; detecting the genetic variant using second isolated nucleic acid molecules in a second sample obtained from the subject at a second time point, according to some embodiments; wherein the first time point is before or after the second time point.

In any of the embodiments herein, the method can be automated. In any of the embodiments herein, an automated system can be configured to implement the method of any of the embodiments disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 depicts a non-limiting exemplary method for isolating nucleic acids, in accordance with some embodiments of the present disclosure.

FIG. 2 depicts a non-limiting exemplary schematic representing a system for isolating nucleic acids, in accordance with some embodiments of the present disclosure.

FIG. 3 depicts a non-limiting example of data indicating the size of isolated nucleic acid fragments, in accordance with some embodiments of the present disclosure.

FIG. 4 depicts a non-limiting example of data that further indicates the size of isolated nucleic acid fragments, in accordance with some embodiments of the present disclosure.

FIG. 5 depicts a non-limiting example of data that indicates the size of isolated nucleic acid fragments from a DNA library preparation protocol, in accordance with some embodiments of the present disclosure.

FIG. 6 depicts a non-limiting example of data that indicates the genomic coverage of the isolated nucleic acids, in accordance with some embodiments of the present disclosure.

FIG. 7 depicts a non-limiting example of data that indicates the percentage of excessively short reads from the isolated nucleic acid fragments, in accordance with some embodiments of the present disclosure.

FIG. 8 depicts a non-limiting example of data that indicates the percentage of low quality reads from the isolated nucleic acid fragments, in accordance with some embodiments of the present disclosure.

### DETAILED DESCRIPTION

Nucleic acid isolation methods can be difficult to automate. Traditional nucleic acid isolation methods often rely on the use of phenol and/or chloroform, which poses challenges to automation. Disclosed herein is a method, which may be an automated method, for isolating nucleic acid molecules from a biological sample. In accordance with the method, proteins are removed from a solution containing nucleic acid molecules, prior to isolating the nucleic acid molecules. The timely removal of nucleic acid molecules can circumvent the use of hazardous solvents such as phenols, and can enable the method to be more readily automatable. A substrate and a binding reagent, which can include an alcohol (for example, isopropanol), are added to the solution after the proteins are digested. In doing so, the nucleic acid molecules bind to the substrate (for example, bead having a silica surface, which may be magnetic). The binding reagent may optionally include other solution components, such as a chaotropic agent, which further enhances binding of the nucleic acid molecules to the substrate. The substrate, once bound to the nucleic acid molecules, can then be separated from the remainder of the solution, and optionally washed. The nucleic acid molecules may then be released from the substrate, providing isolated and purified nucleic acid molecules Also described herein is a system, which may be used to perform the methods, particularly these methods may be easily implemented into an automated system that may be used to perform an automated method for isolating nucleic acid molecules.

Nucleic acid isolation methods can be difficult to automate and scale. Many previously used nucleic acid isolation methods rely on the use of phenol and/or chloroform, which can prove difficult to manipulate with automated methods. Phenol and chloroform are thin non-viscous liquids that are difficult for liquid-handling robotics to manipulate with accuracy. Further, phenols are highly corrosive, and working with solutions comprising phenols can subject robotic hardware to damage over time and usage. The methods and systems described herein do not rely on the use of phenols, and in doing so are scalable and amenable to automation.

The methods and systems described in the present disclosure circumvent the use of phenols by removing proteins from the biological sample, prior to the isolation of the nucleic acid molecules. The removal of proteins prior to the isolation of nucleic acid molecules contrasts against existing methods of nucleic acid isolation. By removing the proteins relatively early, the methods and systems described herein can promote the efficient binding of the nucleic acid molecules onto a substrate, because the nucleic acids do not need to compete with other molecules, such as proteins, for substrate binding. As a result, the methods and systems described herein circumvent the use of phenols and are scalable and amenable to automation.

In addition to increased scalability, the early removal of proteins described for the methods and systems herein, can also enable the more efficient isolation of single-stranded DNA (ssDNA) from biological samples, relative to existing nucleic acid isolation protocols. The isolated ssDNA can comprise ultrashort ssDNA, which can range between 20 and 100 nucleotides in length. The isolation of ssDNA, including ultrashort ssDNA, can be used as a diagnostic or prognostic tool in both a clinical and non-clinical setting. For example, given the recent discovery from Cheng et al., Plasma contains ultrashort single-stranded DNA in addition to nucleosomal cell-free DNA, iScience, vol. 25, no. 104554(2022 ) that human plasma comprises ultrashort ssDNA, in addition to nucleosomal cell-free DNA, the ability to isolate ssDNA directly from samples can be used to efficiently identify ssDNA biomarkers correlated with potential disease symptoms and outcomes. The early removal of proteins as described by the methods and systems herein enables the processing of isolated ssDNAs for both clinical and non-clinical applications, in addition to rendering nucleic acid isolation methods more automatable.

Disclosed herein is a method of isolating nucleic acid molecules comprising: removing proteins from a solution comprising nucleic acid molecules; adding a substrate and a binding agent comprising an alcohol to the solution after removing the proteins from the solution to bind the nucleic acid molecules to the substrate; and separating the substrate bound to the nucleic acid molecules from a remainder of the solution. In some instances, the method can comprise adding a chaotropic agent to the solution. In some instances, the alcohol can comprise isopropanol. In some instances, the substrate can comprise beads, and the beads can be magnetic beads. In some instances, the method of isolating the nucleic acid molecules can be automated, and/or an automated system can be configured to implement the method of isolating the nucleic acid molecules.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The term “agent” refers to a compound, molecule, reagent, ion, or other substance that provides the associated effect. For example, chaotropic agent refers to any substance that causes a chaotropic effect.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

### Methods for Isolating Nucleic Acids

The methods and systems described herein can isolate nucleic acid molecules from a sample with high efficiency by removing proteins from the biological sample prior to binding the nucleic acid molecule to a substrate (such as beads) using a binding reagent comprising an alcohol. Before adding a substrate to the sample, proteins are removed from a solution comprising the nucleic acid molecules. The removal of proteins from the solution can be facilitated, for example, by adding, prior to the protein removal, a proteinase to the solution, which can digest (i.e. degrade) proteins in the solution. Optionally, a detergent can be added along with the proteinase. Additionally or alternatively, proteins may be removed from the solution by precipitating the proteins. For example, proteins may be precipitated by adding a salt, chaotropic agent, or anti-chaotropic agent to the solution. The binding reagent may further include polyethylene glycol (PEG) or a polysaccharaide and/or a chaotropic agent (such as a chaotropic salt). The chaotropic agent in the binding buffer may be the same chaotropic agent as the chaotropic agent used to precipitate the proteins in the prior step, or a different chaotropic agent. Optionally, the substrate and the binding reagent can be added simultaneously to the sample. Once the nucleic acid molecules are bound to the substrate, the substrate may be removed from the solution, for example by centrifugation or filtration. The nucleic acid molecules may then be released from the substrate, for example by mixing the nucleic-acid bound substrate with water or low salt buffer. Examples of low salt buffers can include buffers comprising Tris-HCl or Tris-EDTA (TE).

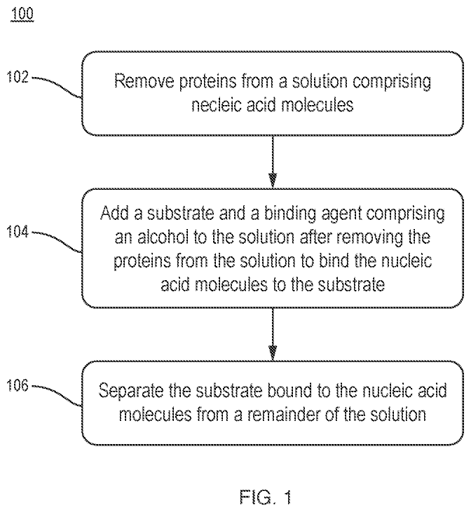

FIG. 1 shows an exemplary schematic showing a general process 100 for isolating nucleic acid molecules from a biological sample. The method of isolating nucleic acid molecules can include: removing proteins from a solution comprising nucleic acid molecules (102); adding a substrate and a binding reagent comprising an alcohol to the solution after removing the proteins from the solution to bind the nucleic acid molecules to the substrate (104); and separating the substrate bound to the nucleic acid molecules from a remainder of the solution (106).

The biological sample can derive from (e.g. be obtained or collected from) a subject (e.g. patient). The subject can be human. The human subject can have a condition or a disease, or be suspected of having a condition or disease, such as cancer. The human subject can also be treated with a therapeutic intervention, such as, but not limited to, a pharmaceutical or a biotechnological intervention. The human subject can be healthy, i.e. free of diagnosed conditions or diseases.

The biological sample can be a liquid sample. The solution can derive from the biological sample. In the case of a liquid sample, a portion of the liquid sample can be removed, e.g. aliquoted, and then combined with a buffer for lysis, such as an extraction buffer, to generate a solution comprising nucleic acids derived from the liquid sample. The solution comprising nucleic acids derived from the biological sample can comprise plasma, whole blood, buffy coat, saliva, serum, sputum, stool, cerebrospinal fluid, or a combination thereof. The solution derived from the sample can comprise nucleic acid molecules. The liquid sample can comprise cell-free DNA (cfDNA).

The solution can comprise nucleic acid molecules. The nucleic acid molecules can comprise single-stranded DNA (ssDNA), double-stranded DNA (dsDNA), and/or RNA. In some instances, the RNA can be mRNA. In some instances, the ssDNA can comprise ultrashort ssDNA, which can range in length between 20 and 100 nucleotides. In some instances, the DNA can be cell-free DNA. Ultrashort ssDNA can be cell-free DNA (cfDNA) and can derive from human plasma. Ultrashort ssDNA can comprise functional genomic elements, such as, but not limited to, promoters, exons, and introns. The nucleic acid molecules can also comprise non-tumor cfDNA and ctDNA, or any combination thereof. The isolated nucleic acid molecules can also comprise a mixture of tumor nucleic acid molecules and non-tumor nucleic acid molecules. In some instances, the sample can comprise a liquid biopsy sample, and the tumor nucleic acid molecules can derive from a ctDNA fraction of the liquid biopsy sample, whereas the non-tumor nucleic acid molecules can derive from a non-tumor cfDNA fraction of the liquid biopsy sample.

At 102 in FIG. 1, proteins are removed from a solution comprising nucleic acid molecules. In some instances, the methods disclosed herein can further comprise adding a proteinase to the solution prior to removing the proteins from the solution. The proteinase can be proteinase K or trypsin. In some instances, the methods disclosed herein can further comprise adding a detergent to the solution prior to removing the proteins from the solution. The detergent can comprise, for example, nonyl phenoxypolyethoxylethanol (NP-40), radio-immunoprecipitation assay (RIPA), sodium dodecyl sulfate (SDS), ammonium-chloride-potassium (ACK), or p-(2,4,4-trimethylpentan-3-yl)phenyl ether (i.e. Triton). In some instances, removing proteins from the solution can comprise precipitating the proteins.

Precipitating the proteins can comprise adding a salt or chaotropic agent to the solution. The chaotropic agent can be a chaotropic salt. The chaotropic agent can be sodium chloride, urea, guanidine thiocyanate, lithium acetate, or sodium thiocyanate. Precipitating the proteins can also comprise centrifuging the proteins into a pellet. After centrifugation, the resulting supernatant comprising the nucleic acid molecules can be isolated from the pellet comprising the proteins, for example by pipetting off the supernatant.

At 104 in FIG. 1, a substrate and a binding reagent comprising an alcohol are added to the solution after removing the proteins from the solution, to bind the nucleic acid molecules to the substrate. Exemplary alcohols that may be included in the binding reagent includes isopropanol and ethanol. In some implementations, the binding reagent comprises isopropanol. In some implementations, the binding reagent comprises ethanol. The binding reagent causes the nucleic acid molecules to bind to the substrate. In some implementations, the binding reagent includes a chaotropic agent. Exemplary chaotropic agents include ammonium ions, potassium ions, sodium ions, lithium ions, magnesium ions, calcium ions, guanidium ions, fluoride ions, sulfate ions, phosphate ions, acetate ions, chloride ions, bromide ions, nitrate ions, chlorate ions, thiocyanate ions, or any combination thereof. The binding reagent can also comprise polyethylene glycol (PEG) and/or a polysaccharide (such as dextran).

In some instances, the substrate can comprise beads. The beads can comprise solid phase reversible immobilization (SPRI) beads, silica beads and/or magnetic beads. The substrate can also comprise or can be coated with silica. In some instances, such as in the case of magnetic beads, the substrate can comprise susceptibility to magnetization, i.e. be ferromagnetic.

At 106 in FIG. 1, the substrate bound to the nucleic acid molecules is separated from a remainder of the solution. The substrate can comprise properties that are conducive to the substrate's separation from the remainder of the solution. In such instances, the substrate bound to the nucleic acid molecules can be separated from the remainder of the solution by applying a magnetic field. After the substrate bound to the nucleic acid molecules has been separated from the remainder of the solution, the nucleic acid molecules can be separated from the substrate. In some instances, the nucleic acids molecules can be separated from the substrate by dissolving the nucleic acid molecules in a solvent. The solvent can be a solution comprising of molecules that can bind well to nucleic acid molecules, such as water, a Tris-EDTA (TE) buffer solution, or some other aqueous solution. Upon eluting the nucleic acid molecules, the substrate may still be bound to the source of the magnetic field, e.g. a magnet. Once the substrate is no longer bound to the nucleic acid molecules, the substrate can be disposed.

In another example, the substrate bound to the nucleic acid molecules can be separated from the remainder of the solution by centrifugation. The solution comprising nucleic acid molecules, can be mixed with a substrate, such as, silica beads, and a binding buffer that includes an alcohol, such as isopropanol. The substrate can bind the nucleic acid molecules but not the remaining sample contents, such as the protein molecules.

Centrifugation can separate the substrate-bound nucleic acid molecules from the remaining sample contents. In some instances, the nucleic acid molecules can be separated from the substrate by dissolving the nucleic acid molecules in a solvent. The solvent can be a solution comprising of molecules that can bind well to nucleic acid molecules, such as water, a Tris-EDTA (TE) buffer solution, or some other aqueous solution. Once the substrate is no longer bound to the nucleic acid molecules, the substrate can be disposed.

### Samples

The disclosed methods and systems may be used with nucleic acid molecules extracted, obtained from, or derived from any of a variety of samples (also referred to herein as specimens) comprising nucleic acids (e.g., DNA or RNA) that are collected from a subject (e.g., a patient).

The biological sample can be a liquid sample. The solution can derive from the biological sample. In the case of a liquid sample, a portion of the liquid sample can be removed, e.g. aliquoted, and then combined with a buffer for lysis, such as an extraction buffer, to generate a solution comprising nucleic acids derived from the liquid sample. The solution comprising nucleic acids derived from the biological sample can comprise, but is not limited to, a tumor sample, a biopsy sample (e.g., a liquid biopsy), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom).

The liquid sample can comprise cell-free DNA (cfDNA).

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the disclosed methods may further comprise analyzing nucleic acid molecules extracted, obtained from, or derived from a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from liquid biopsy samples containing DNA or RNA from certain tissues.

The nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, single-stranded DNA (ssDNA) or fragments thereof, ultrashort ssDNA or fragments thereof, non-tumor cell-free DNA (cfDNA) and circulating tumor DNA (ctDNA). cfDNA is comprised of fragments of DNA that are released from normal and/or cancerous cells during apoptosis and necrosis, and circulate in the blood stream and/or accumulate in other bodily fluids. ctDNA is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

The nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the sample may comprise a tumor fraction or a non-tumor fraction. In some instances, the tumor fraction of the sample may constitute a sample metric. In some instances, the tumor fraction can be the amount of ctDNA divided by the amount of cfDNA. In some instances, the sample may comprise a tumor fraction of at least 0.001%, at least 0.01%, at least 0.1%, at least 1%, at least 5%, at least 10%, at least 15%, at least 20%, at least 25%, at least 30%, at least 35%, at least 40%, at least 45%, at least 50 %, at least 55%, at least 60%, at least 65%, at least 70%, at least 75%, at least 80%, at least 85%, or at least 90%. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content or the tumor fraction of the sample. For example, a sample having a lower tumor content or tumor fraction can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Systems for Isolating Nucleic Acid Molecules

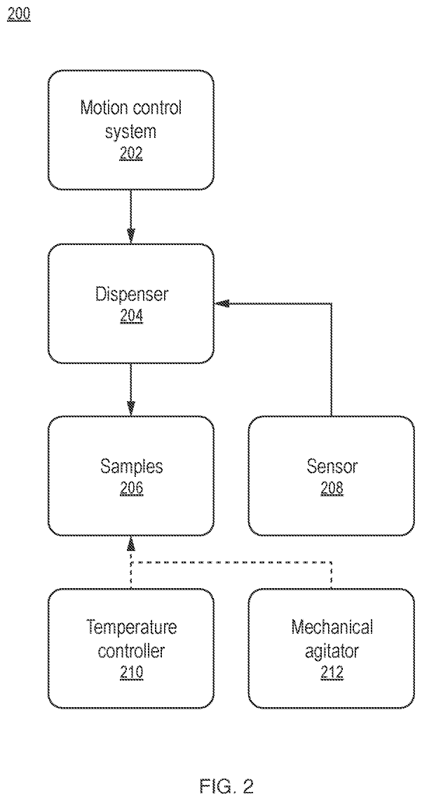

The methods described herein may be performed using a system configured to isolate nucleic acid molecules, such as system 200 represented in FIG. 2. The system may be an automated system, which is configured to automatically perform the disclosed methods. The automated system may be a modular system, where components of the modular system can be substituted, added, and/or removed, for different experimental outputs. Some example modular system components are listed below, e.g. a module for mechanical agitation or a module for temperature control. The automated system can comprise a robotic movement system 202, a dispenser 204, the samples 206, and a sensor 208 that monitors the amount of the samples 206. The automated system can comprise liquid handling robotics (i.e., a liquid handler). Alternatively, the automated system can lack liquid handling robotics. In some implementations the system includes liquid handling robotics and a further system configured to manipulate the sample, for example to bring beads to a liquid reagent.

In some implementations, the automated system includes an instrument that can move beads, such as, but not limited to, silica, magnetic, and/or SPRI beads. For example, the instrument may move the beads to different system components to contact the beads with one or more reagents. This instrument may be, for example, in lieu of a liquid handler than contacts a reagent with the beads by moving the reagents through the system. In another implementation, the system includes both a liquid handler and the instrument that can move the beads. The system can comprise a programmable magnetic module that can manipulate reagents housed in a rotating platform. In such an implementation, the height of the magnetic module's position, the speed of the magnetic module's movement, and the magnetic field strength emanated from the magnetic module, can be programmed. The magnetic module can be used to remove beads from a solution housed on the rotating platform. Similarly, the magnetic module can be used to add beads to a solution housed on the rotating platform. The rotating platform can be rotated in conjunction with the movement of the magnetic module such that the beads can be transferred from one solution housed on the rotating platform to another solution housed on the rotating platform. The solution being manipulated by the magnetic module can be moved from a well-plate on the rotating platform to another well-plate on the rotating platform.

In some implementations, the automated system includes one or more liquid handlers (i.e., a liquid handling robot). The liquid handling robot can comprise a modular tool head, such as, but not limited to, a pipette, syringe, and/or an acoustic droplet ejection-based system) that can dispense allotted volumes of liquids, such as the samples. The dispenser can also be motorized, and the motorization of the dispenser can control the speed and/or volume with which the dispenser dispenses liquids. The dispenser may be situated on a robotic movement system, such as, but not limited to, a gantry or a selective-compliance-articulated robot arm (SCARA), and the robotic movement system can move according to Cartesian-based or quaternion-based coordinates. Similarly, the dispenser can move according to at least one degree of freedom. The dispenser can also comprise multiple pipetting heads, and the pipette heads can be organized in a matrix, where each element of the matrix is a pipette head. The liquid handling robotics can comprise closed-loop error correction methods, such as, but not limited to, a proportional-integrative-derivative (PID)-based and/or proportional-integrative (PI) control loops, based on a sensor that can monitor the amount of liquid being handled. The liquid handling robotics can comprise systems of controlling temperatures of biological samples, such as, but not limited to, thermocycles and/or incubators. The liquid handling robotics can also comprise systems of mechanical agitation, such as, but not limited to, a shaking module, e.g. a vortex shaker, platform shaker, orbital shaker, and/or an incubator shaker. The liquid handling robotics can also comprise a centrifuge system. In some instances, the liquid handling robotics can automate sample placements into, but not limited to, the shaking module, the centrifugation system, or a platform for controlling temperatures.

Also disclosed herein are systems designed to implement any of the disclosed methods for isolating nucleic acids in a sample from a subject. The systems may comprise, e.g., one or more processors, and a memory unit communicatively coupled to the one or more processors and configured to store instructions that, when executed by the one or more processors, cause the system to: remove proteins from a solution comprising nucleic acid molecules, add a substrate and a binding reagent comprising an alcohol to the solution after removing the proteins from the solution to bind the nucleic acid molecules to the substrate; and separate the substrate bound to the nucleic acid molecules from a remainder of the solution.

The automated system can comprise: a) one or more processors; and b) a memory communicatively coupled to the one or more processors and configured to store instructions that, when executed by the one or more processors, cause the system to perform any of the methods or parts of the methods, disclosed herein.

The automated system can also comprise a non-transitory computer-readable storage medium storing one or more programs, the one or more programs comprising instructions, which when executed by one or more processors of the system, can cause the system to perform any of the methods or parts of the methods, disclosed herein.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

The disclosed systems may be used for isolating nucleic acids in any of a variety of samples as described herein (e.g., a tissue sample, biopsy sample, hematological sample, or liquid biopsy sample derived from the subject).

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Applications of Isolating Nucleic Acids

Nucleic acid molecules isolated according to the methods and systems disclosed herein can be further analyzed with additional methods. The additional methods can comprise biotechnological applications, such as, but not limited to, sequencing of the nucleic acid molecules, and/or clinical applications, such as, but not limited to, the identification of disease biomarkers.

In some instances, the methods disclosed herein can further comprise: providing the nucleic acid molecules obtained from a sample from a subject; ligating one or more adapters onto one or more nucleic acid molecules from the nucleic acid molecules; amplifying the one or more ligated nucleic acid molecules from the nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; and receiving, at one or more processors, sequence read data for the plurality of sequence reads.

The methods used to sequence the nucleic acid molecules can comprise specific components and protocols. The one or more adapters can comprise amplification primers, flow cell adaptor sequences, substrate adapter sequences, or sample index sequences. In some instances, the captured nucleic acid molecules can be captured from the amplified nucleic acid molecules by hybridization to one or more bait molecules. The one or more bait molecules can comprise one or more nucleic acid molecules, each comprising a region that is complementary to a region of a captured nucleic acid molecule. Amplifying nucleic acid molecules can comprise performing a polymerase chain reaction (PCR) amplification technique, a non-PCR amplification technique, or an isothermal amplification technique. The sequencing can comprise use of a massively parallel sequencing (MPS) technique, whole genome sequencing (WGS), whole exome sequencing, targeted sequencing, direct sequencing, Nanopore sequencing technique or Sanger sequencing technique. The sequencing can comprise massively parallel sequencing, and the massively parallel sequencing technique can comprise next generation sequencing (NGS). Accordingly, the sequencer can comprise a next generation sequencer.

In some instances, the sequencing of the isolated nucleic acid molecules according to the disclosed methods can be used to determine genetic variants by assessing loci in at least 1, at least 2, at least 3, at least 4, at least 5, at least 6, at least 7, at least 8, at least 9, at least 10, at least 20, at least 30, at least 40, or more than 40 gene loci.

In some instances, the sequencing of the isolated nucleic acid molecules according to the disclosed methods can be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1,XPO1, XRCC2, ZNF217, or ZNF703 gene locus, or any combination thereof.

Similarly, in some instances, the sequencing of the isolated nucleic acid molecules according to the disclosed methods can be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRØ, PD-L1, PI3Kδ, PIGF, PTCH, RAF, RANKL, RET, ROS1, SLAMF7, VEGF, VEGFA, or VEGFB gene locus, or any combination thereof.

In some instances, the disclosed methods may further comprise one or more of the following: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (viii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods for isolating nucleic acids may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

The disclosed methods for isolating nucleic acids may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

The disclosed methods for isolating nucleic acids may be used to select a subject (e.g., a patient) for a clinical trial based on the sequences determined for one or more gene loci. In some instances, patient selection for clinical trials based on, e.g., the sequences at one or more gene loci, may accelerate the development of targeted therapies and improve the healthcare outcomes for treatment decisions.

The disclosed methods for isolating nucleic acids may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

The targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

The disclosed methods for isolating nucleic acids may be used in treating a disease (e.g., a cancer) in a subject. For example, in response to determining a variant using any of the methods disclosed herein, an effective amount of an anti-cancer therapy or anti-cancer treatment may be administered to the subject.

The disclosed methods for isolating nucleic acids may be used for monitoring disease progression or recurrence (e.g., cancer or tumor progression or recurrence) in a subject. For example, in some instances, the methods may be used to determine variants in a first sample obtained from the subject at a first time point, and used to determine variants in a second sample obtained from the subject at a second time point, where comparison of the first determination of variants and the second determination of variants allows one to monitor disease progression or recurrence. In some instances, the first time point is chosen before the subject has been administered a therapy or treatment, and the second time point is chosen after the subject has been administered the therapy or treatment.

The disclosed methods may be used for adjusting a therapy or treatment (e.g., an anti-cancer treatment or anti-cancer therapy) for a subject, e.g., by adjusting a treatment dose and/or selecting a different treatment in response to a change in the determination of variants.

The nucleic acid molecules isolated in accordance with the methods disclosed herein can be sequenced for genetic variants. In some instances, the variants determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

The disclosed methods for identifying variants from the isolated nucleic acid molecules may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for variants as part of a genomic profiling process (or inclusion of the output from the disclosed methods for variants as part of the genomic profile of the subject) can improve the validity of, e.g., disease detection calls and treatment decisions, made on the basis of the genomic profile by, for example, independently confirming the presence of variants in a given patient sample.

A genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. A genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

The methods disclosed herein can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length.

Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; I aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100x or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500 ×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertio-s-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981“, “Identification of Common Molecular Subsequen”es”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (197“) “A General Method Applicable to the Search for Similarities in the Amino Acid Sequence of Two Prote”ns”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite-or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU).

Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B.L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e-6. The likelihood of specific mutations occurring at many sites in, for example, a typical multigenic cancer genome panel can be orders of magnitude higher.

These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011;21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011;21(6):952-660). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequenci-g—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### EXEMPLARY EMBODIMENTS

The following embodiments are exemplary and are not intended to limit the scope of any invention disclosed or claimed herein.

Embodiment 1. A method of isolating nucleic acid molecules comprising:
removing proteins from a solution comprising nucleic acid molecules;
adding a substrate and a binding reagent comprising an alcohol to the solution after removing the proteins from the solution to bind the nucleic acid molecules to the substrate; and
separating the substrate bound to the nucleic acid molecules from a remainder of the solution.

Embodiment 2. The method of embodiment 1, wherein the nucleic acid molecules are derived from a liquid sample.

Embodiment 3. The method of embodiment 1 or embodiment 2, wherein the nucleic acid molecules comprise single-stranded DNA, double-stranded DNA, and/or RNA.

Embodiment 4. The method of embodiment 3, wherein the RNA is mRNA.

Embodiment 5. The method of any of embodiments 1 to 4, wherein the method further comprises adding a proteinase to the solution prior to removing proteins from the solution.

Embodiment 6. The method of embodiment 5, wherein the proteinase is proteinase K or trypsin.

Embodiment 7. The method of any one of embodiments 1 to 6, wherein the method further comprises adding a detergent to the solution prior to removing the proteins from the solution.

Embodiment 8. The method of embodiment 7, wherein the detergent comprises nonyl phenoxypolyethoxylethanol (NP-40) buffer, radio-immunoprecipitation assay (RIPA) buffer, sodium dodecyl sulfate (SDS), ammonium-chloride-potassium (ACK) buffer, or p-(2,4,4-trimethylpentan-3-yl)phenyl ether buffer.

Embodiment 9. The method of any one of embodiments 1-8, wherein removing proteins from the solution comprises precipitating the proteins.

Embodiment 10. The method of embodiment 9, wherein precipitating the proteins comprises adding a chaotropic agent to the solution.

Embodiment 11. The method of embodiment 10, wherein the chaotropic agent is a chaotropic salt.

Embodiment 12. The method of embodiment 10 or embodiment 11, wherein the chaotropic agent is sodium chloride, urea, guanidine thiocyanate, lithium acetate, urea, or sodium thiocyanate.

Embodiment 13. The method of any one of embodiments 9-12, wherein precipitating the proteins comprises centrifuging the proteins into a pellet.

Embodiment 14. The method of embodiment 13, further comprising separating a supernatant comprising the nucleic acid molecules from the pellet.

Embodiment 15. The method of any one of embodiments 1-14, wherein the binding reagent comprises a chaotropic agent.

Embodiment 16. The method of embodiment 15, wherein the chaotropic agent comprises ammonium ions, potassium ions, sodium ions, lithium ions, magnesium ions, calcium ions, guanidium ions, fluoride ions, sulfate ions, phosphate ions, acetate ions, chloride ions, bromide ions, nitrate ions, chlorate ions, thiocyanate ions, or any combination thereof.

Embodiment 17. The method of any one of embodiments 1-16, wherein the binding reagent comprises a polysaccharide or polyethylene glycol (PEG).

Embodiment 18. The method of embodiment 17, wherein the binding reagent comprises the polysaccharide, and wherein the polysaccharide is dextran.

Embodiment 19. The method of any one of embodiments 1-18, wherein the alcohol comprises isopropanol.

Embodiment 20. The method of any one of embodiments 1-19, wherein the alcohol comprises ethanol.

Embodiment 21. The method of any one of embodiments 1-20, wherein the substrate comprises beads.

Embodiment 22. The method of embodiment 21, wherein the beads are solid phase reversible immobilization (SPRI) beads.

Embodiment 23. The method of embodiment 21 or embodiment 22, wherein the beads are silica beads.

Embodiment 24. The method of any of embodiments 21-23, wherein the beads are magnetic beads.

Embodiment 25. The method of embodiment 24, wherein separating the substrate bound to the nucleic acid molecules from the remainder of the solution comprises applying a magnetic field to the magnetic beads.

Embodiment 26. The method of any one of embodiments 1-25, wherein the substrate comprises or is coated with silica.

Embodiment 27. The method of any one of embodiments 1-26, further comprising separating nucleic acid molecules from the substrate after the substrate has been separated from the remainder of the solution.

Embodiment 28. The method of embodiment 27, wherein the nucleic acids molecules are separated from the substrate by dissolving the nucleic acid molecules in a solvent.

Embodiment 29. The method of embodiment 28, wherein the solvent is water or a Tris-EDTA (TE) buffer solution.

Embodiment 30. The method of any one of embodiments 1-29, wherein the solution comprises plasma, whole blood, buffy coat, saliva, serum, sputum, stool, or cerebrospinal fluid.

Embodiment 31. The method of any one of embodiments 1-30, further comprising:
providing the nucleic acid molecules obtained from a sample from a subject; ligating one or more adapters onto one or more nucleic acid molecules from the nucleic acid molecules;
amplifying the one or more ligated nucleic acid molecules from the nucleic acid molecules;
capturing amplified nucleic acid molecules from the amplified nucleic acid molecules; sequencing, by a sequencer, the captured nucleic acid molecules to obtain a plurality of sequence reads that represent the captured nucleic acid molecules; and
receiving, at one or more processors, sequence read data for the plurality of sequence reads.

Embodiment 32. The method of embodiment 31, wherein the one or more adapters comprise amplification primers, flow cell adaptor sequences, substrate adapter sequences, or sample index sequences.

Embodiment 33. The method of embodiment 31 or 32, wherein the captured nucleic acid molecules are captured from the amplified nucleic acid molecules by hybridization to one or more bait molecules.

Embodiment 34. The method of embodiment 33, wherein the one or more bait molecules comprise one or more nucleic acid molecules, each comprising a region that is complementary to a region of a captured nucleic acid molecule.

Embodiment 35. The method of any one of embodiments 31-34, wherein amplifying nucleic acid molecules comprises performing a polymerase chain reaction (PCR) amplification technique, a non-PCR amplification technique, or an isothermal amplification technique.

Embodiment 36. A method of sequencing nucleic acid molecules, comprising:
isolating the nucleic acid molecules according to the method of any one of embodiments 1-30; and
sequencing, using a sequencer, the isolated nucleic acid molecules.

Embodiment 37. The method of any of embodiments 31-36, wherein the sequencing comprises use of a massively parallel sequencing (MPS) technique, whole genome sequencing (WGS), whole exome sequencing, targeted sequencing, direct sequencing, Nanopore sequencing technique or Sanger sequencing technique.

Embodiment 38. The method of embodiment 37, wherein the sequencing comprises massively parallel sequencing, and the massively parallel sequencing technique comprises next generation sequencing (NGS).

Embodiment 39. The method of any one of embodiments 31-38, wherein the sequencer comprises a next generation sequencer.

Embodiment 40. A method of detecting a genetic variant, comprising:
sequencing the isolated nucleic acid molecules according to any one of embodiments 31-39 to obtain a plurality of sequence reads; and
calling, using one or more processors, the genetic variant based on the plurality of sequence reads.

Embodiment 41. A method of detecting the presence of cancer, comprising:
detecting a genetic variant according to the method of embodiment 40, wherein the genetic variant is indicative of a cancer.

Embodiment 42. A method for monitoring cancer progression or recurrence in a subject, the method comprising:
detecting a genetic variant using first isolated nucleic acid molecules in a first sample obtained from the subject at a first time point according to the method embodiment 40;
detecting the genetic variant using second isolated nucleic acid molecules in a second sample obtained from the subject at a second time point;
wherein the first time point is before or after the second time point.

Embodiment 43. A method for monitoring cancer progression or recurrence in a subject, the method comprising:
detecting a genetic variant using first isolated nucleic acid molecules in a first sample obtained from the subject at a first time point;
detecting the genetic variant using second isolated nucleic acid molecules in a second sample obtained from the subject at a second time point according to the method embodiment 40;
wherein the first time point occurs prior to the second time point.

Embodiment 44. The method of embodiment 42 or 43, wherein a cancer treatment is administered to the subject after the first time point and before the second time point.

Embodiment 45. The method of any one of embodiments 40-44, further comprising transmitting the report to a healthcare provider.

Embodiment 46. The method of embodiment 45, wherein the report is transmitted via a computer network or a peer-to-peer connection.

Embodiment 47. The method of any one of embodiments 1-46, wherein the method is automated.

Embodiment 48. An automated system configured to implement the method of any one of embodiments 1-47.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

The following examples further demonstrate to one skilled in the art how to make and use the methods and systems described herein, and are not intended to limit the scope of the claimed invention.

### Example 1

The following examples make reference to three nucleic acid isolation protocols.

One nucleic acid isolation protocol is referred to as the “standard protocol”. The standard protocol comprises turning on a centrifuge, and pre-setting the centrifuge to 4° C. A water bath is also turned on, and set to 60° C. Thawed plasma samples are then transferred to at least one 15 mL tube. A 100-1200 μL 8-channel adjustable pipet is then used to slowly pipette 3 mL of cfDNA binding solution per sample well. The 100-1200 μL 8-channel adjustable pipet is then used to pipette 1 mL of cfDNA wash solution per sample well. The 100-1200 μL 8-channel adjustable pipet is then used to pipette 2 mL of freshly prepared 80% ethanol solution per sample well. The 100-1200 μL 8-channel adjustable pipet is then used to prepare 0.5 mL of freshly prepared 80% ethanol solution per sample well. 80 μL of ctDNA elution buffer is then pipetted into the center of the sample well. All plates are then sealed until ready for use. Once the centrifuge has reached 4° C temperature, the pooled plasma is spun at 7000 rcf for 20 minutes. 75 μL of proteinase K is then added to each of an empty 15 mL tube, wherein an empty 15 mL tube is allocated for each sample. The tubes comprising proteinase K are then stored on ice, until the plasma is added. Once centrifugation is complete, the plasma samples are placed on ice and transferred from each 15 mL tube to the corresponding 15 mL tube containing the proteinase K. Care is taken to avoid transferring the pelleted cellular debris at the bottom of the tubes. The tubes are then well mixed, by inverting the tubes several times. 250 μL of SDS detergent is then added to the plasma, and the resulting solution is then mixed again via inversion. The tubes are then incubated in the water bath at 60° C for 20 minutes. The samples on ice are then cooled for 5 minutes. The samples are then centrifuged briefly to bring all plasma to the bottom of the tube. A 1-10 mL pipette is then used to aspirate 2.5 mL of plasma, and the plasma is dispensed into a plate. The remaining plasma is then dispensed into another plate, to generate a duplicate sample set.

MyOne Silane beads are then vigorously vortexed into a homogenous mixture. The beads are then visually inspected such that they are not clumped on the bottle surface. 37 μL of beads are then added to each sample well in both plates. The samples are then analyzed by the appropriate instrumentation.

Another nucleic acid isolation protocol is a phenol-chloroform extraction-based protocol (“PCI protocol”). The PCI protocol comprises spinning the plasma down at 7000×g for 20 min at 4° C. Then, the supernatant is transferred to a new 15 ml tube. 120 μL of 5 M NaCl, 100 μL of 500 mM EDTA, 150 μL of 20% SDS, and 100 μL of 20 mg/mL proteinase K are then added to the 15 mL tube, and the tube's contents are incubated at 60° C. for 30 minutes. 1 mL of digested plasma is then transferred into five 2 mL tubes. An equal volume of phenol: chloroform: isoamyl alcohol in a 25:24:1 ratio is then added to the 2 mL tubes, and the contents of the tubes are vortexed. The tubes' contents are then spun at 16000×g for 15 minutes, at room temperature. The aqueous phase is then transferred to a new tube, for each of the tubes, and an equal volume of chloroform: isoamyl alcohol is added to each tube. The tubes are then vortexed, and then spun down at 16000×g for 15 minutes at room temperature. The aqueous phase of each tube's contents are then each transferred to a new tube, and 0.1× volume of 3 M sodium acetate (pH 5.2) and 1× volume of isopropanol are added to each tube. The tubes are then left to precipitate at-20 oC, overnight. The tubes are then centrifuged at 16000×g for 30 minutes at room temperature. The pellets of each centrifuged tube's contents are then washed with 70% ethanol, twice. The contents of the tube are then resuspended in 10 μL of TE buffer, and all extractions deriving from a common sample are recombined.

Another nucleic acid isolation protocol adheres to the methods disclosed herein, and is referred to as the “disclosed protocol”. The disclosed protocol comprises placing every 5 mL of plasma sample on ice. 120 μL of 5 M NaCl, 100 μL 500 mM EDTA, and 100 μL 20 mg/mL proteinase K is added to each plasma sample. The contents are then mixed via inversion of the plasma sample(s). 150 μL of 20% SDS detergent is then added to each solution comprising the plasma sample, and each solution is then removed from ice. The solutions comprising the plasma samples are then incubated at 60° C overnight. 400 mL of DNA/RNA buffer is then added to each of the plasma samples, and the samples are then vortexed. The samples are then incubated on ice for three minutes. The samples are then spun at 7000 RCF at 4° C for 30 minutes. The supernatants are then transferred to new tubes and PBS solution is added to bring the final volume of each tube to 6 mL. Lysis and binding buffer comprising isopropanol and silane beads are then added to the solutions comprising the samples. The solution in each tube is then washed in wash buffer, and 80% ethanol is added. The nucleic acids are then eluted in 150 μL of elution buffer.

### Example 2

Plasma from four healthy subjects were acquired, and nucleic acid molecules were isolated from the plasma. For each healthy subject or donor, the nucleic acid amounts deriving from three different nucleic acid isolation protocols are shown: traces 302 and 402 depict data from the standard protocol as described in Example 1, traces 304 and 404 depict data from the PCI protocol as described in Example 1, and traces 306 and 406 depict data from the disclosed protocol as described in Example 1. The lengths of the nucleic acid molecules were then quantified using capillary electrophoresis.

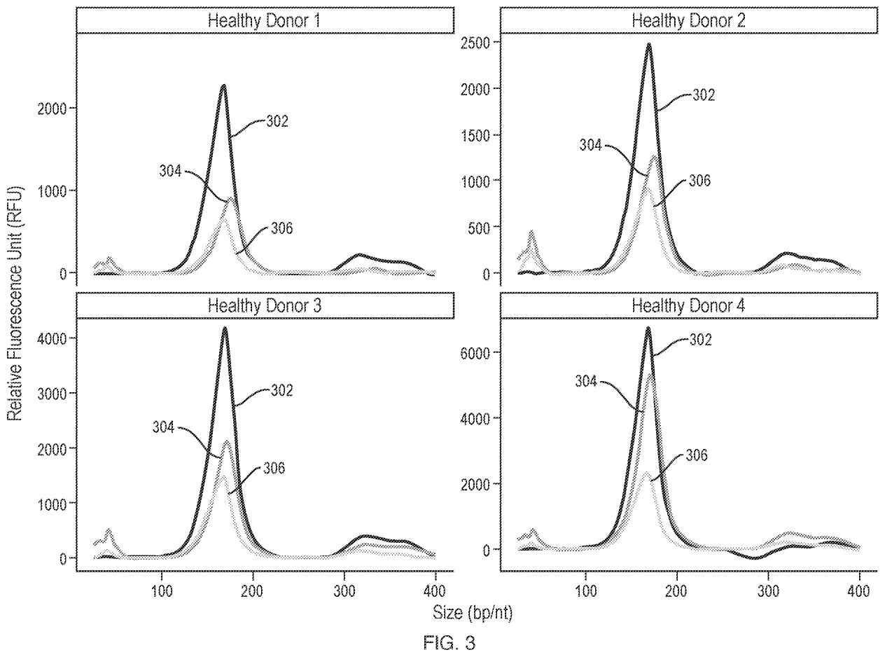

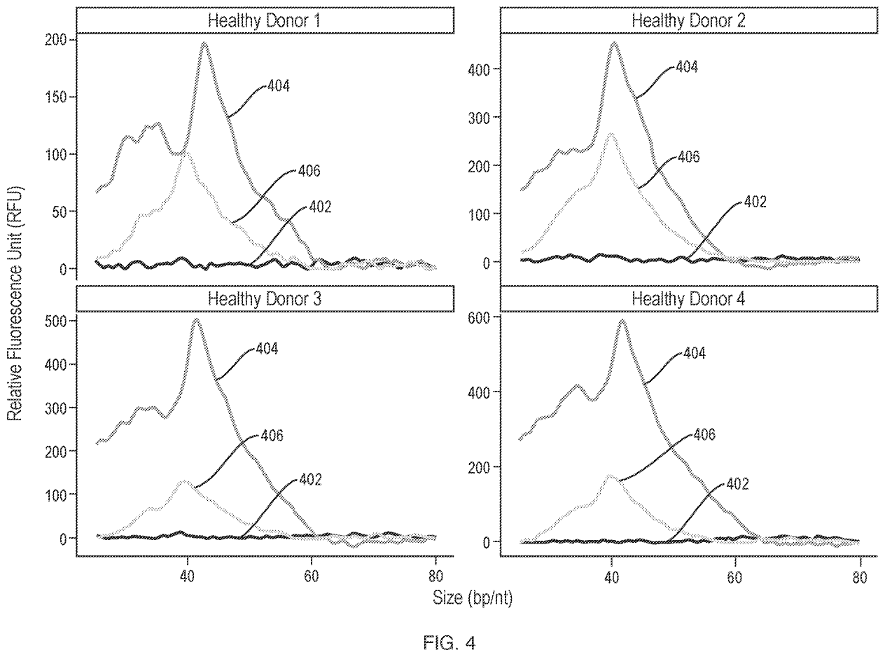

FIG. 3 and FIG. 4 illustrate the sizes of isolated nucleic acids. Both FIG. 3 and FIG. 4 show data deriving from the plasma of four healthy subjects or donors. The x-axes depict the size of the isolated nucleic acid fragments, whereas the y-axes depict the amount of the nucleic acid fragments in terms of artificial fluorescence units. FIG. 4 shows the same data as FIG. 3, but with the x-axes domain limited from 0 to 80 bp/nt, to emphasize the data regarding the smaller isolated nucleic acids. For all nucleic acid isolation protocols and for all subjects, the majority of the isolated nucleic acids range between 100 and 200 bp/nt in size.

In general, as depicted in FIG. 3, the standard protocol provides the highest yields of nucleic acids ranging between 100 and 200 bp/nt, followed by the PCI protocol, followed by the disclosed protocol. In contrast, as depicted in FIG. 4, the PCI protocol provides the highest yield of isolated nucleic acids under 80 bp/nt in size, followed by the disclosed protocol, followed by the standard protocol.

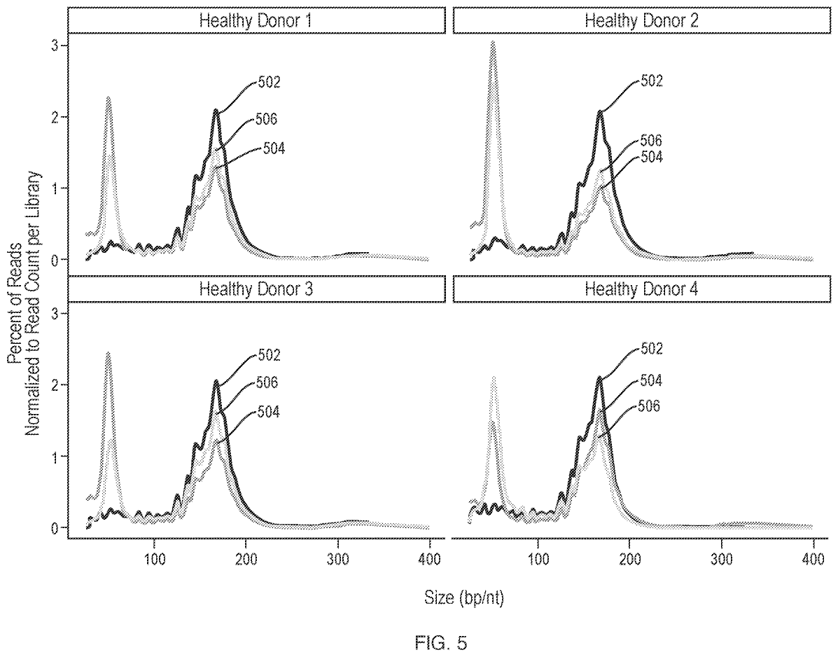

FIG. 5 also depicts examples of data that illustrate the size of isolated nucleic acids, but in the context of a genomic library prep for an Illumina NovaSeq next-generation sequencing platform. The nucleic acid molecules were derived after being subject to a genomic library prep kit, which comprised a 5′ kinase, a 3′ phosphatase, and a ligase. Like in FIG. 3 and FIG. 4, the data depicted in FIG. 5 derive from four healthy donors or subjects, and the x-axes depict the size of the isolated nucleic acid fragments, but unlike in FIG. 3 and FIG. 4, the y-axes depict the percentage of reads normalized to the read counts per library. Like the traces in FIG. 3 and FIG. 4, the traces in FIG. 5 depict nucleic acid amounts deriving from three different nucleic acid isolation protocols: trace 502 depicts data deriving from the standard protocol as described in Example 1, trace 504 depicts data deriving from the PCI protocol as described in Example 1, and trace 506 depicts data deriving from the disclosed protocol as described in Example 1. Of note, trace 502 lacks any short reads under 100 bp/nt for all the subjects depicted, whereas traces 504 and 506 possess a relatively high number of reads under 100 bp/nt. In other words, the PCI protocol and the disclosed protocol can extract small-sized nucleotide fragments that other nucleic acid isolation protocols cannot. The small-sized nucleotide fragments captured by the protocols used to generate traces 504 and 506 may comprise single-stranded nucleic acids, such as, but not limited to, ssDNA. Nucleic acid isolation protocols comprising phenol chloroform, such as the protocol used to generate trace 504, are not, however, scalable. Phenol chloroform is a thin and corrosive liquid that is not amenable to manipulation by liquid handling robotics. A salting out process, such as the protocol used to generate trace 506, is suitable for high-throughput automation, while still capable of isolating small nucleic acid fragments from genomic sequencing library preparations.

### Example 3

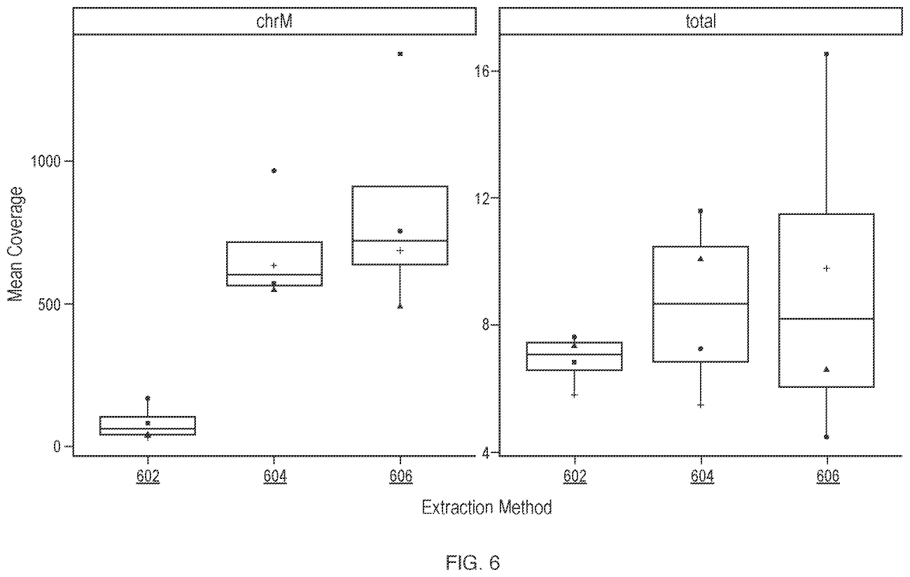

The isolated nucleic acid molecules were sequenced on a next-generation sequencing platform, and the quality of the sequencing reads were assessed, by quantifying various metrics. FIG. 6 depicts the mean genomic coverage of the reads. The glyphs in FIG. 6 depict nucleic acid amounts deriving from three different nucleic acid isolation protocols: glyph 602 depicts data deriving from the standard protocol as described in Example 1, glyph 604 depicts data deriving from the PCI protocol as described in Example 1, and glyph 606 depicts data deriving from the disclosed protocol as described in Example 1. The plot on the left side of FIG. 6 depicts the sequencing coverage of the mitochondrial genome, for the three different nucleic acid isolation protocols, whereas the plot on the right side of FIG. 6 depicts the sequencing coverage of all the chromosomes of the nuclear genome, for the three different nucleic acid isolation protocols. For both the mitochondrial and the nuclear genomes, the nucleic acid isolation protocols used to generate the data for glyphs 604 and 606 provide better genomic coverage than the nucleic acid isolation protocol used to generate the data for glyph 602.

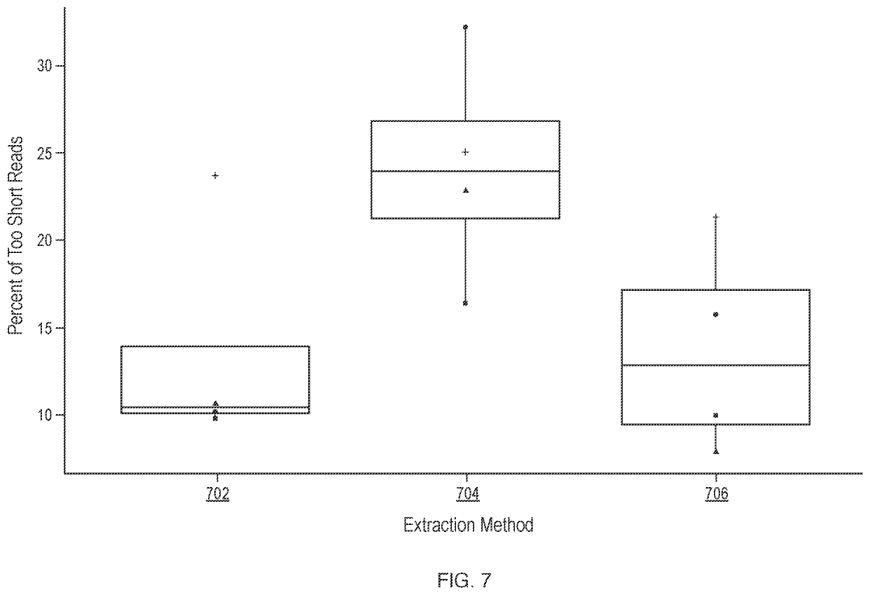

FIG. 7 depicts the percentage of sequencing reads that are considered to be too short for some downstream analyses. The glyphs in FIG. 7 depict nucleic acid amounts deriving from three different nucleic acid isolation protocols: glyph 702 depicts data deriving from the standard protocol, glyph 704 depicts an isolation protocol deriving from the PCI protocol, and glyph 706 depicts data deriving from the disclosed protocol. The nucleic acid isolation protocol used to generate 704, which comprises the use of phenol chloroform, provides the highest percentage of reads that are too short for the use of some downstream analyses. The standard and disclosed protocols provide the highest percentage of reads that are long enough for the use of some downstream analyses.

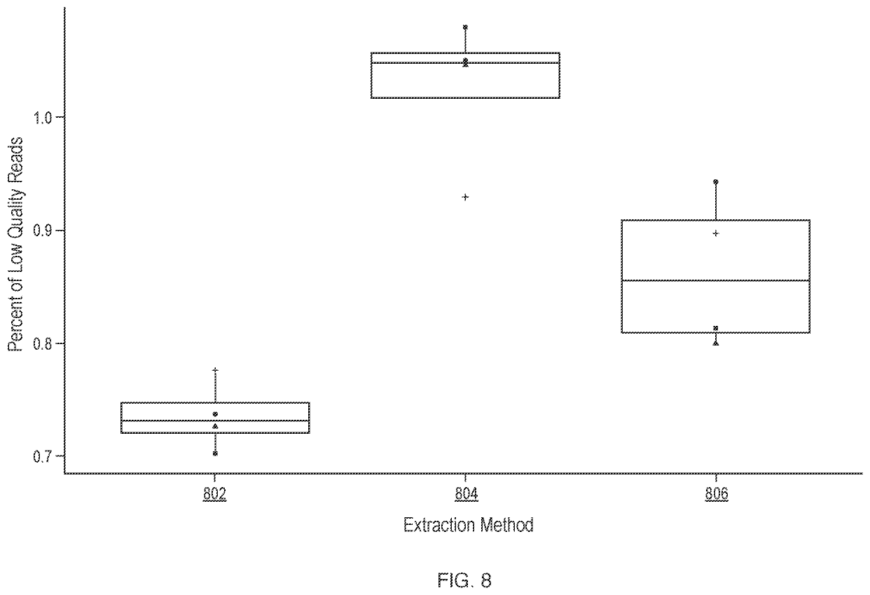

FIG. 8 depicts the percentage of low-quality sequencing reads. Like in FIG. 3 to FIG. 7, the glyphs in FIG. 8 depict nucleic acid amounts deriving from three different nucleic acid isolation protocols: glyph 802 depicts data deriving from the standard protocol, glyph 804 depicts data deriving from the PCI protocol, and glyph 806 depicts data deriving from the disclosed protocol. The nucleic acid isolation protocol used to generate 804, which comprises the use of phenol chloroform, provides the highest percentage of low-quality sequencing reads. The standard and disclosed protocols provide the highest percentage of reads that are of sufficient quality. Given the high yield of nucleic acids derived from the PCI protocol, the data depicted in FIG. 7 and FIG. 8 may suggest that a non-trivial proportion of the yield deriving from phenol chloroform-based nucleic acid isolation protocol may be unideal for the use of some downstream applications.

What is claimed is:

1. A method of isolating nucleic acid molecules comprising:
removing proteins from a solution comprising nucleic acid molecules;
adding a substrate and a binding reagent comprising an alcohol to the solution after removing the proteins from the solution to bind the nucleic acid molecules to the substrate; and
separating the substrate bound to the nucleic acid molecules from a remainder of the solution.

2. The method of claim 1, wherein the nucleic acid molecules are derived from a liquid sample.

3. The method of claim 1, wherein the nucleic acid molecules comprise single-stranded DNA, double-stranded DNA, and/or RNA.

4. The method of claim 1, further comprising adding a proteinase to the solution prior to removing proteins from the solution.

5. The method of claim 4, wherein the proteinase is proteinase K or trypsin.

6. The method of claim 1, further comprising adding a detergent to the solution prior to removing the proteins from the solution.

7. The method of claim 1, wherein removing proteins from the solution comprises precipitating the proteins.

8. The method of claim 7, wherein precipitating the proteins comprises adding a chaotropic agent to the solution.

9. The method of claim 8, wherein the chaotropic agent is a chaotropic salt.

10. The method of claim 8, wherein precipitating the proteins comprises centrifuging the proteins into a pellet.

11. The method of claim 10, further comprising separating a supernatant comprising the nucleic acid molecules from the pellet.

12. The method of claim 1, wherein the binding reagent comprises a chaotropic agent, a polysaccharide, or polyethylene glycol (PEG).

13. The method of claim 1, wherein the alcohol comprises isopropanol or ethanol.

14. The method of claim 1, wherein the substrate comprises beads.

15. The method of claim 14, wherein the beads are solid phase reversible immobilization (SPRI) beads, silica beads, or magnetic beads.

16. The method of claim 15, wherein separating the substrate bound to the nucleic acid molecules from the remainder of the solution comprises applying a magnetic field to the magnetic beads.

17. The method of claim 1, further comprising separating nucleic acid molecules from the substrate after the substrate has been separated from the remainder of the solution.

18. The method of claim 17, wherein the nucleic acids molecules are separated from the substrate by dissolving the nucleic acid molecules in a solvent.

19. The method of claim 1, further comprising:
providing the nucleic acid molecules obtained from a sample from a subject;
ligating one or more adapters onto one or more nucleic acid molecules from the nucleic acid molecules;
amplifying the one or more ligated nucleic acid molecules from the nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules; and
receiving, at one or more processors, sequence read data for the plurality of sequence reads.

20. A method of sequencing nucleic acid molecules, comprising:
isolating the nucleic acid molecules according to the method of claim 1; and
sequencing, using a sequencer, the isolated nucleic acid molecules.
